(19) United States
(12) Patent Application Publication
(10) Pub. No.: US 2021/0241877 A1
(43) Pub. Date: Aug. 5, 2021

(54) INTELLIGENT SITUATIONAL ALARMS AND NOTIFICATIONS FOR DIABETES MANAGEMENT DEVICES

(71) Applicant: Insulet Corporation, Acton, MA (US)

(72) Inventor: Jorge BORGES, Billerica, MA (US)

(21) Appl. No.: 17/150,372

(22) Filed: Jan. 15, 2021

Related U.S. Application Data
(60) Provisional application No. 62/969,792, filed on Feb. 4, 2020.

(51) Int. Cl. G16H 20/17; A61M 5/172

(52) U.S. Cl. CPC G16H 20/17; A61M 2005/14208; A61M 5/1723

(57) ABSTRACT

Disclosed are a device, system, methods and computer-readable medium products that provide techniques to generate a request for user agenda information. The request may be output to a paired device via the wireless communication interface. A request response that includes the user agenda information may be received from the paired device. Diabetes-related alarms and notifications of a diabetes treatment program that correspond to schedule-related information included in the user agenda information may be identified. Adjustments to the alarm and notification parameters related to the identified diabetes-related alarms and notifications of diabetes treatment program according to user preference settings of the diabetes treatment program may be made. Alarms and notifications related to the diabetes treatment program may be implemented according to the adjusted alarm and notification parameters and an alarm or a notification may be output based on the adjusted alarm and notification parameters.

### CROSS-REFERENCE TO RELATED APPLICATIONS

This application claims the benefit of the filing date of U.S. Provisional Application Ser. No. 62/969,792, filed Feb. 4, 2020, the entire contents of which are incorporated herein by reference in their entirety.

### BACKGROUND

A diabetes management device may be an electronic device enables a diabetic and/or their caregivers (e.g., parents) to monitor their blood glucose levels and control delivery of insulin. Diabetes management devices may provide various services to diabetics and their caregivers including notifications (e.g., to remind users to take some action) as well as providing alarms to indicate the potential of harmful conditions or problems with the device or connected devices. The alarms and notifications may occur regularly and can be inconvenient—disturbing and interfering with a diabetes management device user's life while awake or asleep.

Present diabetes management devices may have alarms and/or notifications that may be static, time-based settings of alarms and notifications, and real time user resetting of alarm/notification type and volume.

It would be beneficial and advantageous to have a system or device that addresses these issues facing diabetics and the notifications and alarms provided by diabetes management devices.

### BRIEF DESCRIPTION OF THE DRAWINGS

FIG. 1 illustrates an example of a device operable to provide the example processes and techniques described herein.

FIG. 2 illustrates an example of a process for adjusting alarms and notifications related to a diabetes treatment program.

FIG. 3 illustrates an example of a process executed by a receiving device according to the examples described herein.

FIG. 4 illustrates a functional block diagram of drug delivery system suitable for implementing the example processes and techniques described herein including those described with reference to FIGS. 1-3.

### DETAILED DESCRIPTION

Due to the complicated and dynamic nature of the human body's response to insulin users may end up in a hypoglycemic or hyperglycemic state after being treated with insulin therapy. This outcome is undesirable for many reasons: hypoglycemia creates an immediate risk of a severe medical event (such as a seizure, a coma, or a death) while hyperglycemia creates long term negative health effects as well as the risk of ketoacidosis. Whether a person ends up in one of these states depends on a very complicated combination of many factors and sources of error.

Individuals affected with diabetes have a plethora of complicated decisions to make throughout the day to ensure a user is providing themselves with adequate insulin therapy. An automatic insulin delivery system that utilizes algorithms and/or an artificial pancreas (AP) application is operable to make many insulin delivery and insulin therapy-related decisions for a user so that the user can live their lives as close to the average non-diabetic individual as possible. In order to assist users with making the many insulin delivery and insulin therapy-related decisions, the AP algorithm may generate alarms and notifications via a personal diabetes management (PDM) device, a wearable drug delivery device, a wearable blood glucose sensor, or other devices, such as accessory devices, that assist with management of a diabetes treatment plan.

Often the timing of personal diabetes management alarms and notifications are inconvenient. For example, the alarms or notifications may occur regularly and may occur during social, business or other times, such as while sleeping outside of a user's regular sleep schedule. As a result, the alarms and notifications may be disturbing and interfere with a PDM device user's life while awake or asleep. To improve upon the alarm and notifications, it would be advantageous if devices, processes, computer readable media such as those described herein would provide techniques for identifying when an alarm or notification are to be attenuated or delayed. The disclosed examples provide examples in which PDM device alarm and notification settings (e.g., timing as well as volume and type) are adjusted, for example, based on a user's smart device location, calendar activity, set schedule, or wireless communication availability (e.g., Bluetooth® or 802.11 family of protocols). As described in more detail with reference to the examples, a basic user scenario may enable smart device users, who are also diabetics or caregivers of diabetics, to continue to use their smartphone to manage schedules, create reminders, use map-based applications, and the like, but to also permit a computer application executing with the PDM device to mine data from these activities (e.g., schedule creation, reminder creation, map-based application and the like) on the smartphone to make adjustments to alarms and notifications via the computer application (and using alarm and notification functions or setups) on the PDM device. In an example, the disclosed computer application may be a separate computer application executing on a PDM device that provides information including control information to an AP application. For example, the computer application (i.e., an intelligent situational alarm and notifications application) may be a plug-in to an AP application and may provide commands for setting alarms and/or notifications and/or information (e.g., user agenda information or user preference settings) related to the adjustment of the alarm settings and/or notification settings. The examples are described as implemented via an intelligent situational alarm and notifications application that may be coupled to an AP application.

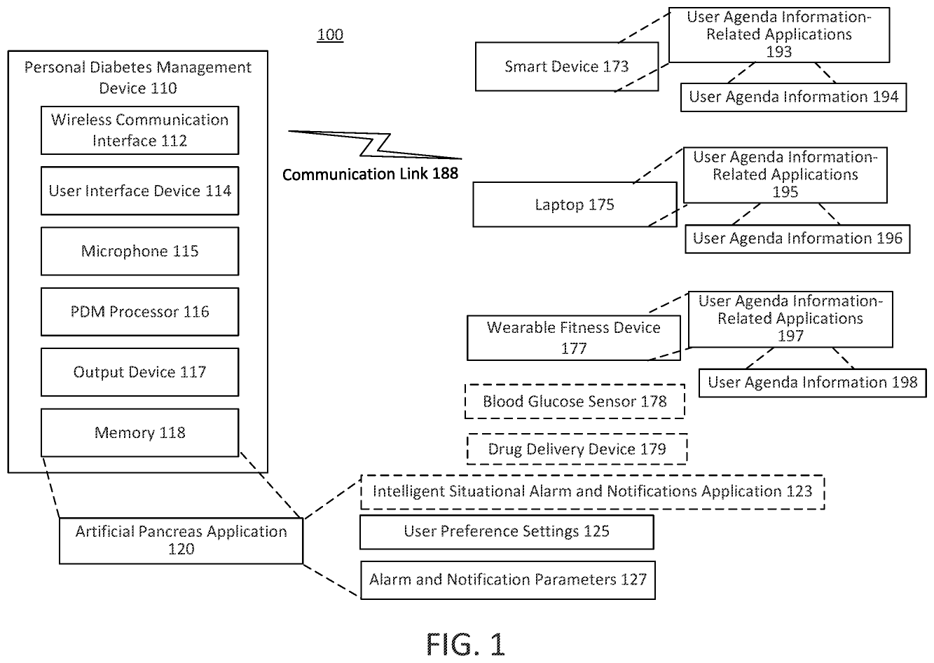

FIG. 1 illustrates an example of a device operable to provide the example processes and techniques described herein. The device 110 may be personal diabetes management device that may have a processor 116, a memory 118, a wireless communication interface 112, and a user interface device 114. Of course, the device 110 may be another device such as a wearable drug delivery device, a wearable blood glucose sensor, or other devices, such as accessory devices, that assist with management of a diabetes treatment plan. For ease of discussion, the device 110 may be referred to as a personal diabetes management (PDM) device.

The user interface device 114 may be buttons coupled to a display, a touchscreen display, or the like that enables a user to interact with and adjust alarm and notification settings as well as other settings. For example, a user, via the user interface device 114, may program the device 110 to respond to an alarm by vibrating via an example of an output device 117 instead of outputting a sound via a speaker (which is another example of an output device 117). In one or more examples, the device 110 may have an output device 117 (such as a speaker, a vibration mechanism, a light or the like), a microphone 115, a user interface device 114, and the like. In an example, the memory 118 may be coupled to the processor 116 and be operable to store programming code and computer applications. The computer applications stored in the memory 118 may include an artificial pancreas (AP) application 120 and other computer applications that, for example, may support implementation of a diabetes treatment plan.

The artificial pancreas application 120 or an intelligent situational alarm and notifications application 123 executing on the processor 116 may communicate with the smart device 173, the laptop 17, or the wearable fitness device 177 via the wireless communication link 188. The artificial pancreas application 120 may incorporate programming code that provides the functionality described with reference to the examples or may be communicatively coupled to another computer application, such as the intelligent situational alarm and notifications application 123, that provides the functionality described in the examples such as requesting, obtaining, or evaluating user agenda information, or other functions as described herein.

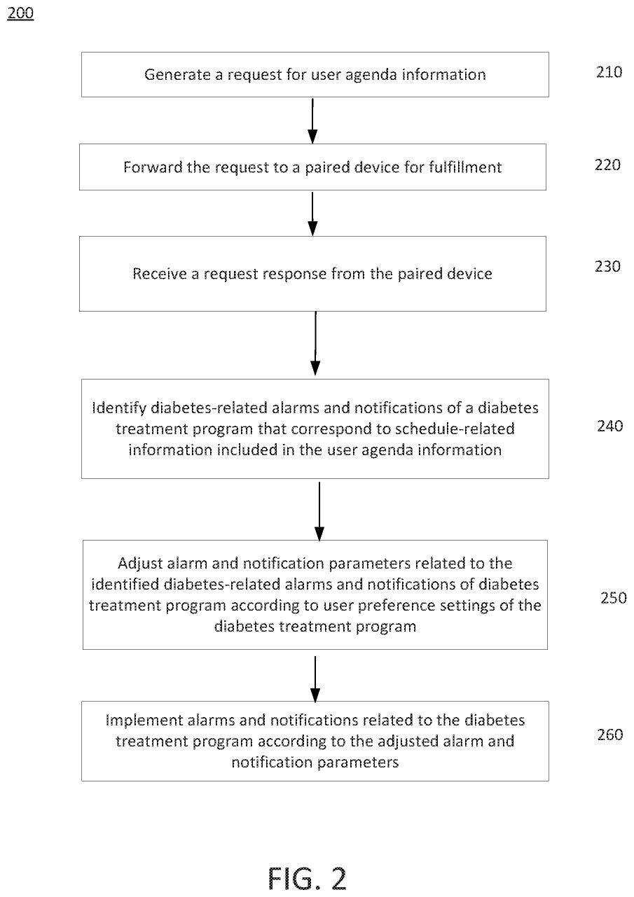

In an example, the artificial pancreas application 120 may be operable to perform functions, including functions to generate a request for user agenda information. The request may include specific access information related to the user agenda information. For example, the specific access information may include passcode, username or other information that enables the personal diabetes management device 110 to gain access to user agenda information-related applications, such as 193, 195 and 197. In an example, user agenda information may include dates and time of business-related events of a user of the requesting device, dates and times of social events, fitness data related to fitness-related activities, dates and times listed in emails, dates and times listed in text messages, location information (e.g., coordinates, street addresses or the like) of the device that receives the request for user agenda information, location names associated with the location information of the device that receives the request for user agenda information, or the like. Each of the respective user agenda information-related applications 193, 195 and 197 may generate or maintain user agenda information 194, 196 and 198, respectively. The request may be output to a paired device, such as the smart device 173 via the wireless communication interface 112 and the wireless communication link 188. The processor 116 may receive, via the wireless communication interface 112, a request response including the user agenda information, such as 194, 196 or 198 from a respective paired device, such as 173, 175 or 177. The processor 116 may be operable to perform processes as described in more detail with reference to the process example of FIG. 2.

In an example, the processor 116 may be operable to establish a connection with one or more of the smart device 173, the laptop 175, or the wearable fitness device 177 via the wireless communication interface 112. A smart device 173 may be a smartphone, a smart wearable device, a smart digital assistant device, or the like. For example, the wireless communication interface 112 may be a Bluetooth® transceiver, or a transceiver operable according to IEEE 802.11 family of communication protocols and may be operable to establish, under control of the processor 116, a wireless communication link 188 with a paired, receiving device, such as the smart device 173, the laptop 175, or the wearable fitness device 177. For example, an artificial pancreas application 120, via the processor, may request via the wireless communication interface 112 a pairing with a receiving device. The pairing request may include an authentication code that indicates to the receiving device, such as 173, 175 or 177, that the request is from a verified device (i.e., the PDM 110). In response to the pairing request, a wireless communication link 188 may be established with the receiving device 173, 175 or 177, which is now a paired device, for receipt of user agenda information.

The processor 116, when executing the artificial pancreas application 120, may be operable to generate a request for information from user preference settings 125 stored in the memory 118. The user preferences settings 125 may include a preset time to generate the request for user agenda information. The processor 116 may identify the preset time in the user preference settings 125 related to the AP application 120 or the intelligent situational alarm and notifications application 123 and may generate a request at the pre-set time. The processor 116 may further be operable to populate the request with indicators from the user preference settings of computer applications executing on the paired device 173, 175 or 177 that have user agenda information (e.g., calendar information, location information, fitness information, or the like as described throughout the disclosure).

In a further example, the artificial pancreas application 120 is operable to communicatively couple via the wireless communication interface 112 to a blood glucose sensor 178, which may be a continuous blood glucose sensor that provides periodic measurements of blood glucose. The blood glucose measurements are received as blood glucose measurement values. The processor 116 may be operable to receive the blood glucose measurement values via the wireless communication interface 112 from the blood glucose sensor 178. For example, the artificial pancreas application 120 may be operable to identify a condition that requires generation of a notification or an alarm based on the received blood glucose measurement values. In response to the identification, the artificial pancreas application 120 may access the adjusted alarm and notification parameters 127. Based on the adjusted alarm and notification parameters 127, the artificial pancreas application 120 may determine a form of the notification or the alarm to be generated in response to the identified condition and output the generated notification or alarm based on the determined form of the notification or the alarm. The form of the generated notification or alarm may be a beeping sound, a synthesized voice, a flashing or continuous light, a pattern of vibrations including a steady vibration, or the like. For example, the output device 117 may be a speaker communicatively coupled to the artificial pancreas application 120 executing on the processor 116. The artificial pancreas application executing on the processor may format the outputted notification or the outputted alarm as synthesized speech.

The artificial pancreas application 120 is further operable to communicatively couple via the wireless communication interface 112 to a wearable drug delivery device 179. The wearable drug delivery device 179 may be physically coupled to a user and provide a drug, such as insulin and/or another drug, to the user in response to commands received via the artificial pancreas application 120. The wearable drug delivery device 179 may provide information regarding status of the device 179, an indication of successful delivery of the drug to the user, or other information. In addition, the wearable drug delivery device 179 may be equipped with alarm or notification devices, such as vibration device, light-emitting-diodes, or a speaker, operable to notify the user of a condition or issue indicated by the alarm or notification.

In an example, the smart device 173 may determine a location of the smart device 173 using various processes. For example, the artificial pancreas application 120 may generate a user agenda information request that is received by the smart device 173. In response to the request, the artificial pancreas application 120 may receive the location data (e.g., from a global positioning system, Wi-Fi communication system, or the like of the smart device 173) as part of the user agenda information. The location data may include a location name. The artificial pancreas application 120 may, in response to a change in location data, modify previously adjusted alarm and notification parameters related to the identified diabetes-related alarms and notifications of a diabetes treatment program.

In some examples, the artificial pancreas application 120 may receive from the paired device, such as 173, 175 or 177, user preferences related to the alarm and notification parameters. For example, the paired device, such as 173, 175 or 177 may have user preferences already set for the particular user agenda information, such as a dentist appoint that has a vibrate alarm or notification set with respect to the dentist appointment. Based on the user preferences associated with the particular user agenda information, the artificial pancreas application may adjust the alarm and notification parameters based on the received user preferences.

An example of a process implemented by a computer application is described in more detail with reference to FIG. 2.

FIG. 2 illustrates an example of a process for adjusting alarms and notifications related to a diabetes treatment program. In the example process 200, a device may generate a request for user agenda information (210). User agenda information may be information provided by computer applications executing on a mobile device of a user, such as a smart phone, a tablet or laptop computing device. The computer applications may include one or more applications such as a calendar application, a fitness application, an email application, a texting application, or a location determination application. Examples of user agenda information may include accelerometer data, gyroscope data, global positioning data, Wi-Fi location data, available Bluetooth devices, dates and times of business-related events of a user of the requesting device, dates and times of social events, fitness data related to fitness-related activities, dates and times listed in emails, dates and times listed in text messages, or location names associated with a location of a device that receives the request from the requesting device. In an example, the person diabetes management device may have user preference settings related to alarm and notifications that depend upon the user agenda information. The person diabetes management device may be operable to, in response to a generate request user preference setting, identify a preset time to generate the request for user agenda information.

In an example, the PDM processor may populate the request with an authentication code that indicates to the paired device that the request for user agenda information is from a verified device.

The request may be forwarded to a paired device for fulfillment (220). A request response may be received from the paired device. The request response may include the requested user agenda information (230). The diabetes-related alarms and notifications of a diabetes treatment program that correspond to schedule-related information included in the user agenda information may be identified by the processor (240). At 250, the processor may adjust alarm and notification parameters related to the identified diabetes-related alarms and notifications of diabetes treatment program according to user preference settings of the diabetes treatment program. The PDM processor may implement alarms and notifications related to the diabetes treatment program according to the adjusted alarm and notification parameters (260). An example of the implementation may include a number of steps that may include identifying a condition that requires generation of a notification. The implementation example may include accessing the adjusted alarm and notification parameters. Based on the adjusted alarm and notification parameters a form of the notification to be generated in response to the identified condition may be determined. The generated notification may, for example be based on the determined form of the notification may be output.

Upon implementation of the alarms and notifications according to the adjusted alarm and notification parameters, the PDM processor may monitor different sensors, such as blood glucose measurement values provided by continuous glucose monitor and other user-related attributes. Based on data provided by the different sensors, the PDM processor may identify a condition that requires generation of a notification. The PDM processor may access the adjusted alarm and notification parameters. Based on the adjusted alarm and notification parameters, the PDM processor may determine a form of the notification to be generated in response to the identified condition. The generated notification may be output based on the determined form of the notification. The form of the notification may be formatted as synthesized speech for output as a spoken notification.

Alternatively, the PDM processor may identify a condition that requires generation of an alarm. The PDM processor may access the adjusted alarm and notification parameters. Based on the adjusted alarm and notification parameters, the PDM processor may determine a form of the alarm to be generated in response to the identified condition. The generated alarm may be output based on the determined form of the alarm. The form of the alarm may be formatted as synthesized speech for output as a spoken alarm.

In specific examples, the user may be a trial lawyer who is often in court. While in the courthouse, the user may prefer any alarm would not be so loud as disrupt the courtroom. Within the bounds of the examples of FIGS. 1 and 2, the user may program the personal diabetes management device to not alarm loudly (perhaps just vibrate) when the user is in the courtroom for a trial. The user's smartphone global positioning system (GPS) receiver or other location system (e.g., cellular or Wi-Fi location determination) may inform the PDM device that the user is at a court house and that the user's smartphone calendar has been blocked off from 11:00 am to 3:00 pm for “Trial”, hence informing AP application that the user is in court for a trial and that any alarms are to be set to vibrate. In addition, once the user leaves the courthouse according to the smartphone GPS, the AP application alarm settings may revert to a previous state.

The user's daily activity routine may be fairly consistent. For example, according to user preferences, the AP application use the user's car Bluetooth to notify him of important events through the car's audio system when he is driving (smartphone GPS indicates rapid movement, plus car's Bluetooth availability to the personal diabetes management device). Similarly, the AP application user preferences may use an indication of the user being at work (via GPS or Wi-Fi location service indications or Bluetooth availability of the user's work computer) and may switch from outputting an audible alarm to outputting a visual alarm (on the personal diabetes management device and his work computer). In another example, a user may have to join an unexpected social activity. The user just before leaving work may get an invitation to join colleagues for a get together after work. The AP application may receive user agenda information, such as an indication from an accelerometer or a GPS application that is interpreted upon evaluation by the AP application as driving. In addition, the smartphone GPS may provide the personal diabetes management device with an indication that the user's is no longer driving, and is now at location, such as a bar. In addition, the user's smartphone microphone can indicate whether the bar is quiet or noisy and further enable the AP application to modify or adjust alarm and notification settings, according to user preference settings. In addition, the AP application may be further operable to modify alarm or notification volume settings in real time or periodically (per a user preference setting, for example) according to changing noise conditions at the bar.

Another example may be when a user is scheduled to attend a business meeting. The user may be in their cubicle and their personal diabetes management alarm settings and/or notification settings prior to the meeting may be set to provide visual alarms via a light (as output device 117) on the user's personal diabetes management and the user's computer (via a wireless communication link). In the example, the user's meeting may be scheduled for 10:00 AM. Because the meeting is in the user's smartphone calendar, the AP application may receive user agenda information indicating the time of the meeting from the user's smartphone and adjust alarm settings based on user preference settings for meetings. The user preference settings may direct the adjustment of the alarm settings from the visual alarm to a vibration alarm at 10:00; however, if the meeting is NOT in her smartphone calendar, the intelligent situational alarm and notifications (ISAN) application may still initiate a change to the alarm and/or notification settings of the personal diabetes management. For example, the smartphone may determine movement of the user (via an accelerometer or pedometer application on the user's smartphone) from the user's cubicle to another location (based on Wi-Fi location information or changes to Bluetooth availability, such as loss of work computer Bluetooth connection to the smartphone) and may adjust alarm and/or notification settings from a visual to vibration in response to the determined movement.

Alternatively, if the user's meeting is suddenly cancelled, or if the user takes their computer to the meeting, the personal diabetes management may continue to have Bluetooth connectivity with their computer and may continue to provide visual alarms or notification—even with no smartphone calendar entry.

In another example of a scenario in which the described examples may be used may be an in game situation. For example, the user may be an active preteen, who plays a lot of soccer. As the user typically does on the weekends, the user may be playing a soccer game and while on the bench, the user's drug delivery device may begin to beep. Since the user, in this example, may typically leave their personal diabetes management device on bench or locker while in a game, he is unable to check his personal diabetes management device to see what the issue is or how severe the condition causing the alarm may be. Alternatively, or in addition, the drug delivery device may be enabled with speech notifications. A speech notification-enabled drug delivery device may be operable to allow a user to tap his drug delivery device to trigger the conversion of the alarm from a beep (or other noise) into speech, which enables a user or a person nearby to understand the issue or the severity of a condition being experienced by the diabetic user.

The computer application described herein is not intended to duplicate smartphone functionality on a personal diabetes management device, but instead take advantage of data already available on the smartphone via the Bluetooth capability of the personal diabetes management device, which may be used to customize or adjust alarms and notifications on the personal diabetes management device.

In an example of a long term implementation, the personal diabetes management device may be operable to continue to mine smartphone data over time and with user permission. The mined data may be evaluated using use artificial intelligence (AI) algorithms to automatically set alarm or notification types and volumes based on a minimal set of user instructions (such as alarm preferences, for example). In a further example, the personal diabetes management device may mine data from health-related apps on the smartphone, or fitness wearables, to set alarms, notifications, insulin-dose reminders, or the like.

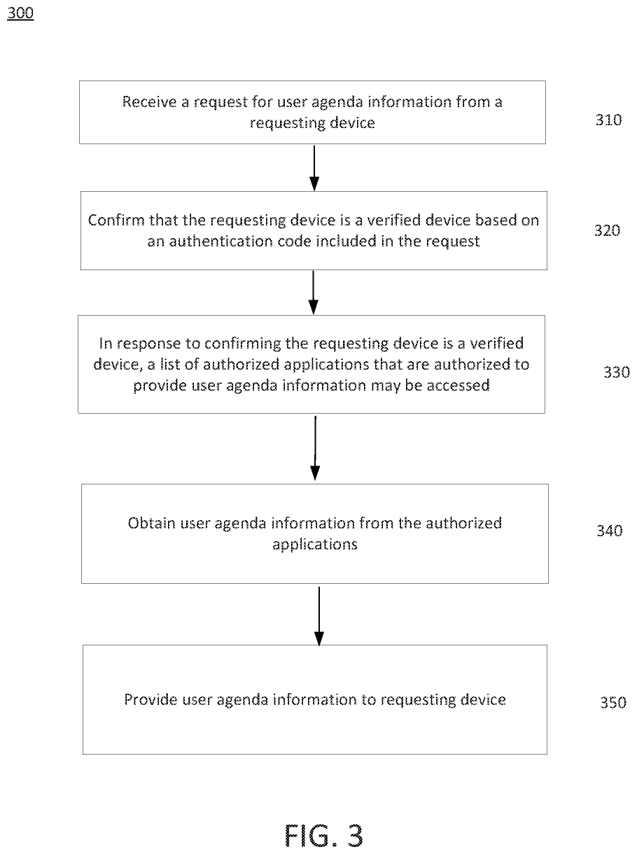

In another example, the smartphone or laptop or other computing device may be referred to more broadly as a receiving device. FIG. 3 illustrates an example of a process executed by a receiving device according to the examples described herein. In the process 300, the receiving device may execute a computer application, such as an intelligent situational alarm and notifications (ISAN) application (such as 123 of FIG. 1) that enables the provision of the user agenda information. For example, the ISAN application executing on the receiving device may receive a request for user agenda information from a requesting device (310). In the example, the requesting device is a personal diabetes management device such as that described with reference to FIGS. 1 and 2. The executing ISAN application being executed by a processor of the receiving device may be operable to confirm that the requesting device is a verified device based on an authentication code included in the request (320). For example, the authentication code may be a user defined code, may be a preset code based on earlier interactions of the PDM and the receiving device, or some other form of authentication code usable to uniquely verify the PDM to the smartphone as a verified device. In an example, the receiving device processor may process the authentication code using a cryptographic algorithm. The cryptographic algorithm may be a hash function or the like. The processor may confirm that the received request was received from a verified device based on an output of the cryptographic algorithm

The process may continue at 330, at which in response to confirming the requesting device is a verified device, a list of authorized applications that are authorized to provide user agenda information may be accessed.

At 340, user agenda information may be obtained from the authorized applications in the list. For example, in response to the request for user agenda information, the user agenda information may be retrieved from the respective authorized applications. In an example, the ISAN application may access user agenda information from one or more applications being executed by the receiving device processor. Examples of the one or more applications may include a calendar application, a fitness application, an email application, a texting application, or a location determination application. In addition, the requesting device may request user agenda information, such as fitness related data, from a wearable fitness device that are not coupled to a smartphone. For example, a requesting device may be operable request and receive fitness-related data (e.g., heart rate, blood oxygen or the like) via a wireless communication link. In a further example, the computer application may be operable, in response to the request for user agenda information, access user agenda information from one or more applications being executed by the processor. The ISAN application may obtain user agenda information from each of the one or more applications for a predetermined time period.

In the example, the receiving device processor may be operable to provide the user agenda information to the requesting device via a wireless communication link (350).

The user agenda information provided to the requesting device may include location data. The location data may, for example, a location name. The application executing on the requesting device may be operable according to user preference settings to provide updates to locations of the receiving device. For example, the ISAN application executing on the receiving device may be operable to provide location data on some predetermined time interval, such as every 15 minutes, 20 minutes or the like. Based on a user preference setting, the ISAN application executing on the receiving device may, in response to a change in location data, provide updated location data including an updated location name to the requesting device.

The requesting device may execute an application that is operable to evaluate the location name and determine whether the location name corresponds to any user preferences related to alarms or notifications of a diabetes treatment program. Alternatively, the location name received in the location data may be different from a location name in an earlier provided location data, which may be interpreted as a change in location of the user.

In the examples of FIGS. 1-3, the example processes may be implemented by programming code, such as the AP application, the intelligent situational alarm and notifications (ISAN) application, or both, that is executed by a processor. The AP application or the ISAN application when executed by a processor may utilize inputs and calculations as described with respect to the foregoing examples.

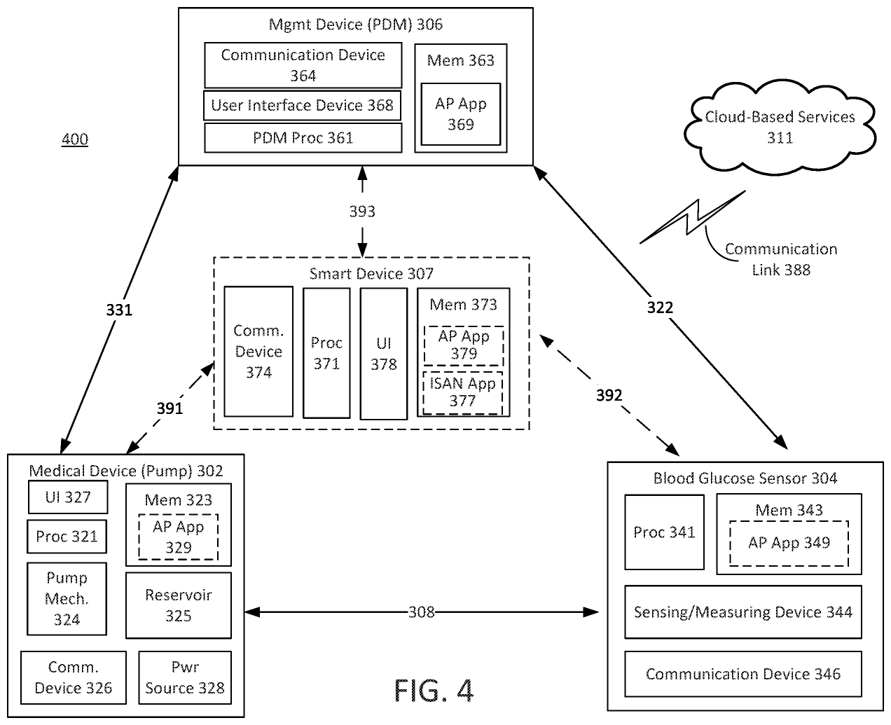

It may be helpful to discuss an example of a drug delivery system that may implement the process example of FIGS. 1-3. FIG. 4 illustrates an example of a drug delivery system suitable for implementing the example processes and techniques described herein including those described with reference to FIGS. 1-3.

The drug delivery system 400 may be operable to implement the process examples illustrated in FIGS. 1-3 by executing an AP application or an intelligent situational alarm and notifications application that includes functionality to generate a request for user agenda information; output the request to a paired device (such as a smart device 307) via the wireless communication interface; receive a request response including the user agenda information from the paired device; identify diabetes-related alarms and notifications of a diabetes treatment program that correspond to schedule-related information included in the user agenda information; adjust alarm and notification parameters related to the identified diabetes-related alarms and notifications of diabetes treatment program according to user preference settings of the diabetes treatment program; implement alarms and notifications related to the diabetes treatment program according to the adjusted alarm and notification parameters; and output an alarm or a notification based on the adjusted alarm and notification parameters.

The drug delivery system 400 may be an automatic drug delivery system that may include a medical device (pump) 302 (also referred to as “a drug delivery device” or “a wearable drug delivery device”), a blood glucose sensor 304 (also referred to as “a continuous glucose monitor” or “a blood glucose measurement device”), and a management device (PDM) 306. The system 400, in an example, may also include a smart device 307, which may be operable to communicate with the PDM 306 and other components of system 400 either via a wired or wireless communication link, such as 391, 392 or 393. In a specific example, the smart device 307 is coupled to the PDM 306 only via a wireless communication link 393, which may be a wireless communication link that utilizes the Bluetooth communication protocol.

In an example, the medical device 302 may be attached to the body of a user, such as a patient or diabetic, and may deliver any therapeutic agent, including any drug or medicine, such as insulin, morphine or the like, to the user. The medical device 302 may, for example, be a wearable device worn by the user. For example, the medical device 302 may be directly coupled to a user (e.g., directly attached to a body part and/or skin of the user via an adhesive or the like). In an example, a surface of the medical device 302 may include an adhesive (not shown) to facilitate attachment to a user.

The medical device 302 may include a number of components to facilitate automatic delivery of a drug (also referred to as a therapeutic agent) to the user. The medical device 302 may be operable to store the drug (i.e., insulin) and to provide the drug to the user. The medical device 302 is often referred to as a pump, or an insulin pump, in reference to the operation of expelling insulin from the reservoir 325 for delivery to the user. While the examples refer to the reservoir 325 storing insulin, the reservoir 325 may be operable to store other drugs or therapeutic agents, such as morphine or the like, that are suitable for automatic delivery.

In various examples, the medical device 302 may be an automatic, wearable drug delivery device. For example, the medical device 302 may include a reservoir 325 for storing the drug (such as insulin), a needle or cannula (not shown) for delivering the drug into the body of the user (which may be done subcutaneously, intraperitoneally, or intravenously), and a pump mechanism (mech.) 324, or other drive mechanism, for transferring the drug from the reservoir 325, through a needle or cannula (not shown), and into the user. The pump mechanism 324 may be fluidly coupled to reservoir 325, and communicatively coupled to the medical device processor 321. The medical device 302 may also include a power source 328, such as a battery, a piezoelectric device, or the like, for supplying electrical power to the pump mechanism 324 and/or other components (such as the processor 321, memory 323, and the communication device 326) of the medical device 302. Although not shown, an electrical power supply for supplying electrical power may similarly be included in each of the sensor 304, the smart device 307 and the management device (PDM) 306.

The blood glucose sensor 304 may be a device communicatively coupled to the PDM processor 361 or pump processor 321 and may be operable to measure a blood glucose value at a predetermined time interval, such as every 5 minutes, or the like. The blood glucose sensor 304 may provide a number of blood glucose measurement values to the AP applications operating on the respective devices (e.g., 329, 349 369, or 379).

The medical device 302 may provide the insulin stored in reservoir 325 to the user based on information (e.g., blood glucose measurement values, predicted future blood glucose measurements, evaluations based on a user request for a bolus, an user interaction with PDM 306, medical device 302, sensor 304 or smart device 307), evaluations of missing blood glucose measurements and the other information provided by the sensor 304, smart device 307, and/or the management device (PDM) 306. For example, the medical device 302 may contain analog and/or digital circuitry that may be implemented as a processor 321 (or controller) for controlling the delivery of the drug or therapeutic agent. The circuitry used to implement the processor 321 may include discrete, specialized logic and/or components, an application-specific integrated circuit, a microcontroller or processor that executes software instructions, firmware, programming instructions or programming code (enabling, for example, the artificial pancreas application (AP App) 329 as well as the process examples of FIGS. 1-3) stored in memory 323, or any combination thereof. For example, the processor 321 may execute a control algorithm, such as an artificial pancreas application 329, and other programming code that may make the processor 321 operable to cause the pump to deliver doses of the drug or therapeutic agent to a user at predetermined intervals or as needed to bring blood glucose measurement values to a target blood glucose value. In an example, the AP application (App) 329 may include programming code that is operable upon execution by the processor 321 to provide the example processes for adjusting or modifying alarm and notification settings, or the like as described with reference to FIGS. 1-3. The user preferences for alarm and notification settings may be programmed, for example, into an artificial pancreas application 329 by the user or by a third party (such as a health care provider, medical device manufacturer, or the like) using a wired or wireless link, such as 331, between the medical device 302 and a management device 306 or other device, such as a computing device at a healthcare provider facility. In an example, the pump or medical device 302 is communicatively coupled to the PDM processor 361 of the management device via the wireless link 331 or via a wireless link, such as 391 from smart device 307 or 308 from the sensor 304. The pump mechanism 324 of the medical device 302 may be operable to receive an actuation signal from the PDM processor 361, and in response to receiving a command signal or an actuation signal, expel insulin from the reservoir 325 based on the commands from an AP application, such as 369.

In an operational example, the AP application 369 executing in the management device 306 may be operable to control delivery of insulin to a user. For example, the AP application 369 may be operable to determine timing of an insulin dose and may output a command signal to the medical device 302 that actuates the pump mechanism 324 to deliver an insulin dose. In addition, the AP application 369 when loaded with programmed code that provides instructions for the functionality of FIGS. 1-3 or the Intelligent Situational Alarm and Notifications Application (not shown) that couples with the AP application 369 to provide the functionality of the examples of FIGS. 1-3.

The other devices in the system 400, such as management device 306, smart device 307 and sensor 304, may also be operable to perform various functions including controlling the medical device 302. For example, the management device 306 may include a communication device 364, a PDM processor 361, and a management device memory 363. The management device memory 363 may store an instance of the AP application 369 that includes programming code, that when executed by the PDM processor 361 provides the process examples described with reference to the examples of FIGS. 1-3. The management device memory 363 may also store programming code for providing the process examples described with reference to the examples of FIGS. 1-3.

The smart device 307 may be, for example, a smart phone, an Apple Watch®, another wearable smart device, including eyeglasses, provided by other manufacturers, a global positioning system-enabled wearable, a wearable fitness device, smart clothing, or the like. Similar to the management device 306, the smart device 307 may also be operable to perform various functions including controlling the medical device 302. For example, the smart device 307 may include a communication device 374, a processor 371, and a memory 373. The memory 373 may store an instance of the AP application 379 and/or an instance of the intelligent situational alarm and notifications (ISAN) application 377 (and such as 123 of FIG. 1) that includes programming code for providing the process examples described with reference to the examples of FIGS. 2 and 3. The memory 373 may also as store programming code and be operable to store data related to the AP application 379 or the intelligent situational alarm and notifications application 377. In an operational example, the AP application 379 may be operable to provide functionality similar to or the same the functionality as described with reference to the instance of the AP application 369.

The sensor 304 of system 400 may be a continuous glucose monitor (CGM) as described above, that may include a processor 341, a memory 343, a sensing or measuring device 344, and a communication device 346. The memory 343 may, for example, store an instance of an AP application 349 as well as other programming code and be operable to store data related to the AP application 349 and process examples described with reference to FIGS. 1-3. The AP application 349 may also include programming code for providing the process examples described with reference to the examples of FIGS. 1-3.

Instructions for determining the delivery of the drug or therapeutic agent (e.g., as a bolus dosage) to the user (e.g., the size and/or timing of any doses of the drug or therapeutic agent) may originate locally by the medical device 302 or may originate remotely and be provided to the medical device 302. In an example of a local determination of drug or therapeutic agent delivery, programming instructions, such as an instance of the artificial pancreas application 329, stored in the memory 323 that is coupled to the medical device 302 may be used to make determinations by the medical device 302. In addition, the medical device 302 may be operable to communicate with the cloud-based services 311 via the communication device 326 and the communication link 388.

Alternatively, the remote instructions may be provided to the medical device 302 over a wired or wireless link (such as 331) by the management device (PDM) 306, which has a PDM processor 361 that executes an instance of the artificial pancreas application 369, or the smart device 307 (via communication link 391), which has a processor 371 that executes an instance of the artificial pancreas application 369 as well as other programming code for controlling various devices, such as the medical device 302, smart device 307 and/or sensor 304. The medical device 302 may execute any received instructions (originating internally or from the management device 306) for the delivery of the drug or therapeutic agent to the user. In this way, the delivery of the drug or therapeutic agent to a user may be automatic.

In various examples, the medical device 302 may communicate via a wireless link 331 with the management device 306. The management device 306 may be an electronic device such as, for example, a smart phone, a tablet, a dedicated diabetes therapy management device, or the like. The management device 306 may be a wearable wireless accessory device. The wireless links 308, 331, 322, 391, 392 and 393 may be any type of wireless link provided by any known wireless standard. As an example, the wireless links 308, 331, 322, 391, 392 and 393 may enable communications between the medical device 302, the management device 306 and sensor 304 based on, for example, Bluetooth®, Wi-Fi®, a near-field communication standard, a cellular standard, or any other wireless optical or radio-frequency protocol.

The sensor 304 may be a glucose sensor operable to measure blood glucose and output a blood glucose value or data that is representative of a blood glucose value. For example, the sensor 304 may be a glucose monitor or a continuous glucose monitor (CGM). The sensor 304 may include a processor 341, a memory 343, a sensing/measuring device 344, and communication device 346. The communication device 346 of sensor 304 may include one or more sensing elements, an electronic transmitter, receiver, and/or transceiver for communicating with the management device 306 over a wireless link 322 or with medical device 302 over the link 308. The sensing/measuring device 344 may include one or more sensing elements, such as a glucose measurement, heart rate monitor, or the like. The processor 341 may include discrete, specialized logic and/or components, an application-specific integrated circuit, a microcontroller or processor that executes software instructions, firmware, programming instructions stored in memory (such as memory 343), or any combination thereof. For example, the memory 343 may store an instance of an AP application 349 that is executable by the processor 341.

Although the sensor 304 is depicted as separate from the medical device 302, in various examples, the sensor 304 and medical device 302 may be incorporated into the same unit. That is, in various examples, the sensor 304 may be a part of the medical device 302 and contained within the same housing of the medical device 302 (e.g., the sensor 304 may be positioned within or embedded within the medical device 302). Glucose monitoring data (e.g., measured blood glucose values) determined by the sensor 304 may be provided to the medical device 302, smart device 307 and/or the management device 306 and may be used to perform the functions and deliver doses of insulin for automatic delivery of insulin by the medical device 302 as described with reference to the examples of FIGS. 1-3.

The sensor 304 may also be coupled to the user by, for example, adhesive or the like and may provide information or data on one or more medical conditions and/or physical attributes of the user. The information or data provided by the sensor 304 may be used to adjust drug delivery operations of the medical device 302.

In an example, the management device 306 may be a computing device operable to manage a personal diabetes treatment plan via an AP application or an algorithm. The management device 306 may be used to program or adjust operation of the medical device 302 and/or the sensor 304. The management device 306 may be any portable electronic, computing device including, for example, a dedicated controller, such as PDM processor 361, a smartphone, or a tablet. In an example, the management device (PDM) 306 may include a PDM processor 361, a management device memory 363, and a communication device 364. The management device 306 may contain analog and/or digital circuitry that may be implemented as a PDM processor 361 (or controller) for executing processes to manage a user's blood glucose levels and for controlling the delivery of the drug or therapeutic agent to the user. The PDM processor 361 may also be operable to execute programming code stored in the management device memory 363. For example, the management device memory 363 may be operable to store an artificial pancreas (AP) application 369 that may be executed by the PDM processor 361. The PDM processor 361 may when executing the artificial pancreas application 369 may be operable to perform various functions, such as those described with respect to the examples in FIGS. 1 and 2. The communication device 364 may be a receiver, a transmitter, or a transceiver that operates according to one or more radio-frequency protocols. For example, the communication device 364 may include a cellular transceiver and a Bluetooth transceiver that enables the management device 306 to communicate with a data network via the cellular transceiver and with the sensor 304 and the medical device 302. The respective transceivers of communication device 364 may be operable to transmit signals containing information useable by or generated by the AP application or the like. The communication devices 326, 346 and 376 of respective medical device 302, sensor 304 and Smart device 307 may also be operable to transmit signals containing information useable by or generated by the AP application or the like.

The medical device 302 may communicate with the sensor 304 over a wireless link 308 and may communicate with the management device 306 over a wireless link 331. The sensor 304 and the management device 306 may communicate over a wireless link 322. The smart device 307, when present, may communicate with the medical device 302, the sensor 304 and the management device 306 over wireless links 391, 392 and 393, respectively. The wireless links 308, 331, 322, 391, 392 and 393 may be any type of wireless link operating using known wireless standards or proprietary standards. As an example, the wireless links 308, 331, 322, 391, 392 and 393 may provide communication links based on Bluetooth®, Wi-Fi, a near-field communication standard, a cellular standard, or any other wireless protocol via the respective communication devices 326, 346 and 364. In some examples, the medical device 302 and/or the management device 306 may include a user interface 327, 378 and 368, respectively, such as a keypad, a touchscreen display, levers, buttons, a microphone, a speaker, a light, a display, or the like, that is operable to allow a user to enter information and allow the management device to output information for presentation to the user. Note that the respective user interface devices 327, 378 and 368 may also be output devices that provide indications of an alarm or notification to the user.

In various examples, the drug delivery system 400 may implement the artificial pancreas (AP) algorithm (and/or provide AP functionality) to govern or control automatic delivery of insulin to a user (e.g., to maintain euglycemia—a normal level of glucose in the blood). The AP application may be implemented by the medical device 302 and/or the sensor 304. The AP application may be used to determine the times and dosages of insulin delivery. In various examples, the AP application may determine the times and dosages for delivery based on information known about the user, such as the user's sex, age, weight, or height, and/or on information gathered about a physical attribute or condition of the user (e.g., from the sensor 304). For example, the AP application may determine an appropriate delivery of insulin based on glucose level monitoring of the user through the sensor 304. The AP application may also allow the user to adjust insulin delivery. For example, the AP application may allow the user to issue (e.g., via an input) commands to the medical device 302, such as a command to deliver an insulin bolus. In some examples, different functions of the AP application may be distributed among two or more of the management device 306, the medical device (pump) 302 or the sensor 304. In other examples, the different functions of the AP application may be performed by one device, such the management device 306, the medical device (pump) 302 or the sensor 304.

As described herein, the drug delivery system 400 or any component thereof, such as the medical device may be considered to provide AP functionality or to implement an AP application. Accordingly, references to the AP application (e.g., functionality, operations, or capabilities thereof) are made for convenience and may refer to and/or include operations and/or functionalities of the drug delivery system 400 or any constituent component thereof (e.g., the medical device 302 and/or the management device 306). The drug delivery system 400—for example, as an insulin delivery system implementing an AP application—may be considered to be a drug delivery system or an AP application-based delivery system that uses sensor inputs (e.g., data collected by the sensor 304).

In an example, one or more of the devices, 302, 304, 306 or 307 may be operable to communicate via a wireless communication link 388 with cloud-based services 311. The cloud-based services 311 may utilize servers and data storage (not shown). The communication link 388 may be a cellular link, a Wi-Fi link, a Bluetooth link, or a combination thereof, that is established between the respective devices 302, 304, 306 or 307 of system 400. The data storage provided by the cloud-based services 311 may store user agenda information related to the user, or the like. In addition, the cloud-based services 311 may process anonymized data from multiple users to provide generalized information related to the various parameters used by the AP application.

In an example, the device 302 includes a communication device 364, which as described above may be a receiver, a transmitter, or a transceiver that operates according to one or more radio-frequency protocols, such as Bluetooth, Wi-Fi, a near-field communication standard, a cellular standard, that may enable the respective device to communicate with the cloud-based services 311. For example, outputs from the sensor 304 or the medical device (pump) 302 may be transmitted to the cloud-based services 311 for storage or processing via the transceivers of communication device 364. Similarly, medical device 302, management device 306 and sensor 304 may be operable to communicate with the cloud-based services 311 via the communication link 388.

In an example, the respective receiver or transceiver of each respective device, 302, 306 or 307, may be operable to receive signals containing respective blood glucose measurement values of the number of blood glucose measurement values that may be transmitted by the sensor 304. The respective processor of each respective device 302, 306 or 307 may be operable to store each of the respective blood glucose measurement values in a respective memory, such as 323, 363 or 373. The respective blood glucose measurement values may be stored as data related to the artificial pancreas algorithm, such as 329, 349, 369 or 379. In a further example, the AP application operating on any of the management device 306, the Smart device 307, or sensor 304 may be operable to transmit, via a transceiver implemented by a respective communication device, 364, 374, 346, a control signal for receipt by a medical device. In the example, the control signal may indicate an amount of insulin to be expelled by the medical device 302.

Various operational scenarios and examples of processes performed by the system 400 are described herein. For example, the system 400 may be operable to implement the process examples of FIG. 1-3.

The techniques described herein for providing functionality to generate a request for user agenda information; output the request to a paired device via the wireless communication interface; receive a request response from the paired device, wherein the request response includes the user agenda information; identify diabetes-related alarms and notifications of a diabetes treatment program that correspond to schedule-related information included in the user agenda information; adjust alarm and notification parameters related to the identified diabetes-related alarms and notifications of diabetes treatment program according to user preference settings of the diabetes treatment program; implement alarms and notifications related to the diabetes treatment program according to the adjusted alarm and notification parameters; and output an alarm or a notification based on the adjusted alarm and notification parameters. For example, the system 400 or any component thereof may be implemented in hardware, software, or any combination thereof. Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

An example provides a process that may be used with any additional algorithms or computer applications that manage blood glucose levels and insulin therapy. Such algorithms may be referred to as an “artificial pancreas” algorithm-based system, or more generally, an artificial pancreas (AP) application, that provides automatic delivery of an insulin based on a blood glucose sensor input, such as that received from a CGM or the like. In an example, the artificial pancreas (AP) application when executed by a processor may enable a system to monitor a user's glucose values, determine an appropriate level of insulin for the user based on the monitored glucose values (e.g., blood glucose concentrations or blood glucose measurement values) and other information, such as user-provided information, such as carbohydrate intake, exercise times, meal times or the like, and take actions to maintain a user's blood glucose value within an appropriate range. The appropriate blood glucose value range may be considered a target blood glucose value of the particular user. For example, a target blood glucose value may be acceptable if it falls within the range of 80 mg/dL to 120 mg/dL, which is a range satisfying the clinical standard of care for treatment of diabetes. Alternatively, in addition, an AP application as described herein may be able to establish a target blood glucose value more precisely and may set the target blood glucose value at, for example, 110 mg/dL, or the like. As described in more detail with reference to the examples of FIGS. 1-4, the AP application may utilize the monitored blood glucose values and other information to generate and send a command to a medical device including, for example, a pump, to control delivery of a dose of insulin to the user, change the amount or timing of future doses, as well as to control other functions, such as the modification of alarm settings and notification settings, user preference settings for alarms and notifications, generation of prompts, receipt of inputs from a user, or the like.

In addition, or alternatively, while the examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed AP application and algorithms may be operable to perform various functions related to open loop operations, such as the generation of prompts identifying the softened upper bound that presented to a user via a user interface. Similarly, a dosage amount of insulin may be received by the AP application or algorithm from a user via a user interface. Other open-loop actions may also be implemented by adjusting user settings or the like in an AP application or algorithm.

Some examples of the disclosed device may be implemented, for example, using a storage medium, a computer-readable medium, or an article of manufacture which may store an instruction or a set of instructions that, if executed by a machine (i.e., processor or microcontroller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of example examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A non-transitory computer readable medium embodied with programming code executable by a processor, and the processor when executing the programming code is operable to perform functions, including functions to:
generate a request for user agenda information;
forward the request to a paired device for fulfillment;
receive a request response from the paired device, wherein the request response includes the user agenda information;
identify diabetes-related alarms and notifications of a diabetes treatment program that correspond to schedule-related information included in the user agenda information;
adjust alarm and notification parameters related to the identified diabetes-related alarms and notifications of diabetes treatment program according to user preference settings of the diabetes treatment program; and
implement alarms and notifications related to the diabetes treatment program according to the adjusted alarm and notification parameters.

2. The non-transitory computer readable medium of claim 1, further embodied with programming code executable by a processor, and the processor when executing the programming code is further operable to perform functions, including functions to:
when generating the request for user agenda information, access user preference settings;
in the user preference settings, identify a preset time to generate the request for user agenda information; and
generate the request for the user agenda information at the pre-set time.

3. The non-transitory computer readable medium of claim 1, further embodied with programming code executable by a processor, and the processor when executing the programming code is further operable to perform functions, including functions to:
populate the request with an authentication code that indicates to the paired device that the request for user agenda information is from a verified device.

4. The non-transitory computer readable medium of claim 1, further embodied with programming code executable by a processor, and the processor when executing the programming code is further operable to perform functions, including functions to:
identify a condition that requires generation of a notification;
access the adjusted alarm and notification parameters;
determine based on the adjusted alarm and notification parameters a form of the notification to be generated in response to the identified condition; and
output the generated notification based on the determined form of the notification.

5. The non-transitory computer readable medium of claim 4, further embodied with programming code executable by a processor, and the processor when executing the programming code is further operable to perform functions, including functions to:
format the form of the notification as synthesized speech for output as a spoken notification.

6. The non-transitory computer readable medium of claim 1, further embodied with programming code executable by a processor, and the processor when executing the programming code is further operable to perform functions, including functions to:
identify a condition that requires generation of an alarm;
access the adjusted alarm and notification parameters;
determine based on the adjusted alarm and notification parameters a form of the alarm to be generated in response to the identified condition; and
output the generated alarm based on the determined form of the alarm.

7. The non-transitory computer readable medium of claim 6, further embodied with programming code executable by a processor, and the processor when executing the programming code is further operable to perform functions, including functions to:
format the form of the alarm as synthesized speech for output as a spoken alarm.

8. A non-transitory computer readable medium embodied with programming code executable by a processor, and the processor when executing the programming code is operable to perform functions, including functions to:
receive a request for user agenda information from a requesting device, wherein the requesting device is a personal diabetes management device;
confirm the requesting device is a verified device based on an authentication code included in the request;
in response to confirming the requesting device is a verified device, access a list of authorized applications that are authorized to provide user agenda information;
obtain user agenda information from the authorized applications; and
provide the user agenda information to the requesting device.

9. The non-transitory computer readable medium of claim 8, further embodied with programming code executable by a processor, and the processor when executing the programming code is further operable to perform functions, including functions to:
process the authentication code using a cryptographic algorithm; and
based on an output of the cryptographic algorithm, confirm that the received request was received from a verified device.

10. The non-transitory computer readable medium of claim 8, further embodied with programming code executable by a processor, and the processor when executing the programming code is further operable to perform functions, including functions to:
in response to the request for user agenda information, access user agenda information from one or more applications being executed by the processor, wherein the one or more applications include a calendar application, a fitness application, an email application, a texting application, or a location determination application;
obtain information-related to the user agenda information from each of the one or more applications for a predetermined time period; and
provide the information-related to the user agenda information to the requesting device.

11. The non-transitory computer readable medium of claim 8, further comprises:
provide location data as part of the user agenda information, wherein the location data includes a location name; and
in response to a change in location data, provide updated location data including an updated location name to the requesting device, wherein the providing of updated location data is based on a user preference setting.

12. The non-transitory computer readable medium of claim 8, wherein the user agenda information includes:
dates and time of business-related events of a user of the requesting device, dates and times of social events, fitness data related to fitness-related activities, dates and times listed in emails, dates and times listed in text messages, or location names associated with a location of a device that receives the request from the requesting device.

13. A device, comprising:
a processor;
a memory coupled to the processor and operable to store programming code, an artificial pancreas application and data;
a wireless communication interface operable to wirelessly communicate with a paired device and communicatively coupled to the processor;
wherein the artificial pancreas application is executable by the processor, wherein the processor, when executing the artificial pancreas application, is operable to perform functions, including functions to:
generate a request for user agenda information;
output the request to a paired device via the wireless communication interface;
receive a request response from the paired device, wherein the request response includes the user agenda information;
identify diabetes-related alarms and notifications of a diabetes treatment program that correspond to schedule-related information included in the user agenda information;
adjust alarm and notification parameters related to the identified diabetes-related alarms and notifications of diabetes treatment program according to user preference settings of the diabetes treatment program;
implement alarms and notifications related to the diabetes treatment program according to the adjusted alarm and notification parameters; and
output an alarm or a notification based on the adjusted alarm and notification parameters.

14. The device of claim 13, wherein the processor, when executing the artificial pancreas application, is further operable to perform functions, including functions to:
in response to a generate request user preference setting, identify a preset time to generate the request for user agenda information; and
populate the request with indicators from the user preference settings of computer applications executing on the paired device that have agenda information included in the user agenda information.

15. The device of claim 13, wherein the processor, when executing the artificial pancreas application, is further operable to perform functions, including functions to:
request a pairing via the wireless communication interface with a receiving device, the request including an authentication code that indicates to the receiving device that the request is from a verified device; and
establish a wireless communication link with the receiving device for receipt of user agenda information.

16. The device of claim 13, wherein:
the wireless communication interface is operable to communicatively couple to a continuous blood glucose sensor,
the processor operable to receive blood glucose measurement values via the wireless communication interface from the continuous blood glucose sensor, and
the processor, when executing the artificial pancreas application, is further operable to perform functions, including functions to:
identify based on the received blood glucose measurement values a condition that requires generation of a notification or an alarm;
access the adjusted alarm and notification parameters;
determine based on the adjusted alarm and notification parameters a form of the notification or the alarm to be generated in response to the identified condition; and
output the generated notification or alarm based on the determined form of the notification or the alarm.

17. The device of claim 13, further comprising:
a speaker communicatively coupled to the processor,
wherein the processor, when executing the artificial pancreas application, is further operable to perform functions, including functions to:
format the outputted notification or the outputted alarm as synthesized speech; and
forward the synthesized speech to the speaker.

18. The device of claim 13, wherein the user agenda information includes:
accelerometer data, gyroscope data, global positioning data, Wi-Fi location data, available Bluetooth devices, dates and times of business-related events of a user of a requesting device, dates and times of social events, fitness data related to fitness-related activities, dates and times listed in emails, dates and times listed in text messages, or location names associated with a location of a device that receives the request from the requesting device.

19. The device of claim 13, wherein the processor, when executing the artificial pancreas application, is further operable to perform functions, including functions to:
receive location data as part of the user agenda information, wherein the location data includes a location name; and
in response to a change in location data, modify the adjusted alarm and notification parameters related to the identified diabetes-related alarms and notifications of diabetes treatment program.

20. The device of claim 13, wherein the processor, when executing the artificial pancreas application, is further operable to perform functions, including functions to:
receive, from the paired device, user preferences related to the alarm and notification parameters; and
adjust the alarm and notification parameters based on the received user preferences.
